(19) United States
(12) Patent Application Publication
(10) Pub. No.: US 2007/0237797 A1
(43) Pub. Date: Oct. 11, 2007

(54) Neural Conduit Agent Dissemination

(75) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(73) Assignee: Gholam A. Peyman, Pittsboro, NC (US)

(21) Appl. No.: 11/278,992

(22) Filed: Apr. 7, 2006

Related U.S. Application Data
(63) Continuation-in-part of application No. 11/277,639, filed on Mar. 28, 2006.

(51) Int. Cl. A61N 1/39; A61K 9/00; A61K 9/127

(52) U.S. Cl. 424/400; 424/450; 607/1; 977/906

(57) ABSTRACT

A method for dissemination of a biocompatible agent using a neural conduit. In one embodiment, agent dissemination is facilitated, for example, by the application of thermal energy, ultrasound energy, radiant energy, electromagnetic energy, or electrical current. As one example, agent may be provided to a sensory organ such as the eye, ear, or nose, for dissemination along the optic nerve to the central nervous system. As another example, agent may be provided at an acupuncture site for dissemination along a peripheral nerve to either a second peripheral nerve site or a central nervous system site.

This application is a Continuation In Part of copending U.S. patent application Ser. No. 11/277,639 filed Mar. 28, 2006, which is expressly incorporated by reference herein in its entirety.

Use of a neural pathway to provide an agent to a proximal and/or distal neural site is disclosed. In one embodiment, providing an agent at a site in the peripheral nervous system (PNS), which includes sensory organs such as the eye, ear, tongue, and nose, provides agent to the central nervous system (CNS). In another embodiment, providing an agent at a site in the central nervous system provides diffuse dissemination and/or channeled dissemination to the peripheral nervous system along neural conduits. In another embodiment, a neural conduit provides an agent to one or more specific areas in the central nervous system and/or peripheral nervous system.

It is known that silicone oil administered intravitreally to an individual for retinal tamponade migrated along the intracranial portion of the optic nerve into the brain. Retrograde transport to the brain from each of the optic nerve and the retina has been reported. It is known that an opioid may be topically administered intranasally and absorbed through the nasal mucosa to relieve migraines, as disclosed in U.S. Pat. No. 5,855,807 which is expressly incorporated by reference herein in its entirety. Efficacy of topical intranasal administration may involve the sphenopalatine ganglion (SPG) of the trigeminal system, located immediately posterior to and immediately above the posterior tip of the middle turbinate behind the nasal mucosa. SPG has sensory, parasympathetic, and sympathetic nerve supplies. It is the major source of parasympathetic innervation to brain vasculature, and its stimulation increases blood brain barrier permeability (Yarnitsky et al., Brain Res, 1018: 236 (2004)).

Such a central and/or peripheral neural conduit is useful for agent administration, delivery, and dissemination. An agent that is capable of providing and/or enhancing a diagnostic and/or therapeutic effect, either directly or indirectly, may be used.

In one embodiment, the agent is administered at a first site in the peripheral nervous system, for example, the sensory system, for action in a second site in the sensory or other peripheral nervous system site, and/or for action in the central nervous system. In another embodiment, the agent is administered at a site within the central nervous system and transported for action to other sites in the central nervous system and/or peripheral nervous system.

As used herein and as generally recognized by one skilled in the art, the central nervous system encompasses the brain and spinal cord. Access to specific regions within the central nervous system may be limited by neuroanatomy and/or neurophysiology. As one example, a neural conduit may be used to provide agents as therapy for Alzheimer's disease to regions of the brain that control thought, memory, and language, functions that are affected in individuals having this disease. Agents may be specifically provided by a neural conduit to sites of amyloid plaques and/or neurofibrillary tangles in the brain. As another example, a neural conduit may be used to provide agents to specific receptors (e.g., agonists and/or antagonists), ion channels, neurotransmitters, etc. that are perturbed in patients with schizophrenia, depression, or other psychiatric disorders.

As used herein and as generally recognized by one skilled in the art, the peripheral nervous system encompasses sensory afferent nerves and motor efferent nerves. Motor efferent nerves further include the somatic nervous system controlling voluntary skeletal muscles, and the autonomic nervous system. The autonomic nervous system has a parasympathetic component to maintain homeostasis, and a sympathetic component to permit a fight or flight response. The inventive method may use any or all of these neural conduits.

In the central nervous system, a unique membranous barrier tightly segregates the brain from the circulating blood. The barrier function is due to the capillaries in the central nervous system that are structurally different from capillaries in other tissues; these structural differences result in a permeability barrier (blood brain barrier, BBB) between blood maintained within these capillaries and the extracellular fluid in brain. In vertebrates, these brain and spinal cord capillaries lack the small pores or fenestrations that allow rapid movement of agents from the circulation into other organs; instead, they are lined with a layer of special endothelial cells that are sealed with tight junctions.

These capillaries make up about 95% of the total surface area of the BBB, and represent the principal route through which chemicals enter the brain. The capillaries have smaller diameters and thinner walls than capillaries in other organs. Because they essentially lack intercellular clefts, pinocytosis functions, and fenestrae, any exchange into or out of these capillaries must pass trans-cellularly (across cells). Therefore, only lipid-soluble agents that can freely diffuse through the capillary endothelial membrane passively cross the BBB.

In addition to its structural barrier aspect, the BBB also has an enzymatic aspect. Agents crossing the capillary endothelial cell membrane are exposed to mitochondrial enzymes that recognize and rapidly degrade most peptides, including naturally occurring neuropeptides.

Segregation of agents from the central nervous system, i.e., outside the BBB, is further reinforced by a high concentration of P-glycoprotein (Pgp) active-drug-efflux-transporter proteins in the capillary endothelial cell luminal membrane. These efflux transporter proteins actively remove a broad range of agents from the cytoplasm of endothelial cells before the agents can cross into the brain parenchyma.

Segregation mechanisms such as these render the brain essentially inaccessible to many agents, including lipid-insoluble (i.e., hydrophilic) compounds, for example, polar molecules and small ions. As a consequence, the therapeutic value of otherwise promising agents is diminished, and cerebral diseases are rendered refractory to therapeutic interventions.

Neurons, specialized cells within the central and peripheral nervous systems that conduct electrochemical impulses termed action potentials, are composed of a cell body that contains the nucleus and other organelles, an axon, and dendrites. An axon is a cytoplasmic extension of the cell body and is controlled by the cell body. Axons can be of considerable length and require a steady transport of materials (e.g., vesicles, mitochondria) from the cell body along its entire length. Transport is driven by proteins, termed kinesins and dyneins, that move along microtubules in the axon. Dendrites are the site of origin of nerve impulses which are then conducted along the axon.

Neuronal transport is the general term for movement of large molecules within cell bodies. Molecules may be moved within a cell (intraneuronal transport) and between cells (interneuronal transport). Neurons efficiently communicate and transport agents to and from the cell body to the axons and dendrites. Both slow and fast transport mechanisms are used. Proteins, such as cytoskeletal structural proteins and many enzymes, are carried by slow axonal transport. Agents required at synapses between nerves are carried by more rapid axonal transport. Different protein populations are transported along axons and dendrites, so the proteins are likely sorted in the cell body into separate and distinctive types of transport vesicles. Chemical communication occurs in both directions. Retrograde transport provides larger materials back from the axons and dendrites to the cell body and is a relatively slower transport process. Anterograde transport provides smaller materials from the cell body to the termini and is a relatively faster process.

Viral-mediated neuronal transport mechanisms have been used in an attempt to target agents into the brain using retrograde transport. Some viruses have evolved an ability to use nerve transport to gain access to the nervous system, which otherwise is well protected against foreign invasion. These neurotrophic viruses, such as polio virus and herpes virus, are typically very specific in the areas of the nervous system that they attack and effect. An adeno-associated viral vector was used to target delivery of a neuroprotective gene to defined neuronal populations. Viral delivery to axon termini in the hippocampus and striatum resulted in viral internalization, retrograde transport, and transgene expression in specific projection neurons in the entorhinal cortex and substantia nigra. Using viral vectors in the nervous system, however, raises practical and safety issues.

Certain carbohydrate-binding proteins such as lectins have been used to transport agents to neurons or other target cells and within neurons via neuronal transport, for putative treatment of neurologically related conditions. Specific lectin compositions are known (e.g., U.S. Patent Application Publication No. 2005/0027119) and include a non-toxic lectin transport entity operably linked to a therapeutic agent so that the agent is capable of being transported to a target. A method for treating a neurological condition includes administering the therapeutic agent and lectin to a patient needing treatment for a neurological condition, with the therapeutic agent operably linked to a non-toxic lectin so that the therapeutic agent is capable of being transported to a target associated with the neurological condition.

Other mechanisms can be used to provide agents using neural conduits to or from the central nervous system. In one embodiment, the inventive method utilizes a localized site in the peripheral nervous system to disseminate the agent at diffuse sites in the central nervous system. As only one example, administering an agent at an ocular site (peripheral nervous system) uses neural conduits to ventricles of the brain and diffusive distribution through the cerebrospinal fluid to provide agent to the brain and/or spinal cord (central nervous system).

In one embodiment, agents to be delivered include, but are not limited to, gene therapy agents contained in a vector, vaccines, peptides, proteins, and drugs such as macrolides, steroids, matrix metalloproteinase (MMP) inhibitors, anti-prostaglandins, non-steroidal anti-inflammatory agents (NSAIDS), antibiotics, antiviral agent, antioxidants, anti-proliferative agents, anti-cell migration agents, anti-angiogenic agents, agents with efficacy in particular regions of the brain, and/or anti-leukotrienes. Non-limiting examples follow, each known to one skilled in the art.

Macrolides include, but are not limited to, tacrolimus (FK506), Cyclosporin A, sirolimus (rapamycin), pimocrolus, ascomycin, everolimus, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, lincosamide, and/or derivatives any of the above (e.g., sirolimus derivatives temsirolimus, AP23573). The concentration of macrolide is such that it is without substantial toxicity to the retina. For example, a concentration of up to 200 μg/ml is efficacious for ocular use and is without substantial toxicity. Macrolides and macrolide analogues as known to one skilled in the art may have neurostimulatory and/or neuroprotective activity.

Steroids include, but are not limited to, triamcinolone (Aristocort®; Kenalog®), betamethasone (Celestone®), budesonide, cortisone, dexamethasone (Decadron-LA®; Decadron® phosphate; Maxidex® and TobraDex® (Alcon), hydrocortisone, methylprednisolone (Depo-Medrol®, Solu-Medrol®), prednisolone (prednisolone acetate, e.g., Pred Forte® (Allergan); Econopred and Econopred Plus® (Alcon); AK-Tate® (Akorn); Pred Mild® (Allergan); prednisone sodium phosphate (Inflamase Mild and Inflamase Forte® (Ciba); Metreton® (Schering); AK-Pred® (Akorn)), fluorometholone (fluorometholone acetate (Flarex® (Alcon); Eflone®), fluorometholone alcohol (FML® and FML-Mild®), (Allergan); Fluor OP®)), rimexolone (Vexol® (Alcon)), medrysone alcohol (HMS®) (Allergan)); Iotoprednol etabonate (Lotemax® and Alrex® (Bausch & Lomb), 11-desoxcortisol, and anacortave acetate (Alcon)).

Antibiotics include, but are not limited to, doxycycline (4-(dimethylamino)-1,4,4a,5,5a,6,11, 12a-octahydro-3,5,10,12,12a-pentahydroxy-6-methyl-1,1-dioxo-2-naphthacenecarboxamide monohydrate, C₂₂H₂₄N₂O₈.H₂O), aminoglycosides (e.g., streptomycin, amikacin, gentamicin, tobramycin, cephalosporins (e.g., beta lactams including penicillin), tetracyclines, amantadine, polymyxin B, amphtotericin B, amoxicillin, ampicillin, atovaquone, azithromycin, bacitracin, cefazolin, cefepime, cefotaxime, cefotetan, cefpodoxime, ceftazidime, ceftizoxime, ceftriaxone, cefuroxime, cephalexin, chloramphenicol, clotimazole, ciprofloxacin, clarithromycin, clindamycin, dapsone, dicloxacillin, erythromycin, fluconazole, gatifloxacin, griseofulvin, isoniazid, itraconazole, ketoconazole, metronidazole, nafcillin, neomycin, nitrofurantoin, nystatin, pentamidine, rifampin, rifamycin, valacyclovir, vancomycin, etc.

Antiviral agents include, but are not limited to, α-interferon, β-interferon, γ-interferon, nucleoside analogues such as acyclovir (acycloguanosine), ganciclovir, foscarnet, zidovudine (azidothymidine), lamivudine[3TC], ribarvirin, amantadine, idoxuridine, dideoxyinosine, and dideoxycytadine.

Anti-proliferative agents include, but are not limited to, carboplatin, 5-fluorouracil (5-FU), thiotepa, etoposide (VP-16), doxorubicin, ifosphophamide, cyclophosphamide, etc.

Anti-prostaglandins include, but are not limited to, indomethacin, ketorolac tromethamine 0.5% ((±)-5-benzoyl-2,3-dihydro-1H-pyrrolizine-1-carboxylic acid, compound with 2-amino-2-(hydroxymethyl)-1,3-propanediol (1:1) (Acular® Allegan, Irvine Calif.), Ocufen® (flurbiprofen sodium 0.03%), meclofenamate, fluorbiprofen, and compounds in the pyrrolo-pyrrole group of non-steroidal anti-inflammatory drugs.

MMP inhibitors include, but are not limited to, doxycycline, TIMP-1, TIMP-2, TIMP-3, TIMP-4, MMP1, MMP2, MMP3, Batimastat, or marimastat.

Anti-angiogenesis agents include, but are not limited to, antibodies to vascular endothelial growth factor (VEGF) such as bevacizumab (Avastin®), rhuFAb V2 (ran ibizumab Lucentis®) (Genentech, South San Francisco Calif.), pegaptanib (Macugen®, Eyetech Pharmaceuticals, New York N.Y.), sunitinib maleate (Sutent®, Pfizer, Groton CT), TNP470, integrin av antagonists, 2-methoxyestradiol, paclitaxel, P38 mitogen activated protein kinase inhibitors, anti-VEGF siRNA (short double-stranded RNA to trigger RNA interference and thereby impair VEGF synthesis); pigment epithelium derived factor(s) (PEDF); Celebrex®; Vioxx®; interferon α; interleukin-12 (IL-12); thalidomide and derivatives such as Revimid™(CC-5013) (Celgene Corporation); squalamine; endostatin; angiostatin; the ribozyme inhibitor Angiozyme® (Sirna Therapeutics); and multifunctional antiangiogenic agents such as Neovastat® (AE-941) (Aeterna Laboratories, Quebec City, Canada).

Agents with efficacy in one or more regions of the brain include, but are not limited to, acetylcholinestrase inhibitors (e.g., tacrine, rivastigmine, metrifonate), L-type calcium channel modulators (isradipine, (R)(+)-Bay K8644, (S)(−)-Bay K8644, (±)-Bay K8644, calcicludine 2, dilantizem, felodipine, FS-2, FPL 64176, nicardipine, (R)(−)-niguldipine hydrochloride, (S)(+)-niguldipine hydrochloride, nifedipine, nimodipine, nitrendipine, S-petasin, phloretin, (±)-verapamil hydrochloride, etc.), nicotinic alpha-7 receptor agonists, inhibitors of phosphodiesterase 10 (PDE10) (e.g. papaverine, inhibitors selected according to the method disclosed in U.S. Patent Application Publication No. 20030032579 to Pfizer), and inhibitors of phosphodiesterase 4 (PDE4) (e.g., AWD 12-281, GW842470). Specific targeting to the brain may be achieved, for example, using endogenous receptor-mediated transport pathways at the BBB as disclosed in Shi and Pardridge, Non-Invasive Targeting to the Brain, PNAS, 97; 7567 (2000); Pardridge, W M, Blood-Brain Barrier Drug Targeting: The Future of Brain Drug Development, Molecular Interventions, 3; 90 (2003); Zhang and Pardridge, Neuroprotection in Transient Focal Brain Ischemia After Delayed Intravenous Administration of Brain-Derived Neurotrophic Factor Conjugated to a Blood-Brain Barrier Drug Targeting System, Stroke, 32; 1379 (2001), etc., as known to one skilled in the art.

Anti-leukotrienes include, but are not limited to, zileuton, genleuton, BAYX1005, MK-886, LY171883, MK-571, cinalukast, montelukast, and pranlukast.

One skilled in the art will appreciate that the above agents are representative only, and that agents under development or in clinical trials may also undergo neural conduit dissemination and are included within the inventive method.

In an embodiment where agent is administered at one or more peripheral nervous system sites, e.g., the eye, nasal mucosa, etc., the agent interacts with microtubules in the axons by which the agent is transported. The microtubules run the length of the axon, providing a system of tracks. Both proteins, e.g., cholera toxin B, and carbohydrates, e.g., the lectin agglutinin, interact with axon microtubules. Protein and/or carbohydrate agents may be administered according to the inventive method.

Neural transport encompasses intraneuronal transport, interneuronal transport, transsynaptic transport, transport from the peripheral nervous system to the central nervous system, transport from one site in the peripheral nervous system to another site (proximal or distal) in the peripheral nervous system, transport from the central nervous system to the peripheral nervous system, and/or transport from one site in the central nervous system to another site (proximal or distal) in the central nervous system. Intraneuronal transport encompasses agent movement within the neuron following its introduction into the neuron. It includes transport from axonal nerve terminals to the cell body (retrograde transport), transport from dendritic nerve terminals to the cell body (retrograde transport), transport from dendritic nerve terminals to axonal nerve terminals, transport from axonal nerve terminals to dendritic nerve terminals, and transport to axonal and dendritic nerve terminals from the cell body (anterograde transport). Interneuronal transport encompasses transsynaptic transport whereby agent moves from one neuron to another across the synaptic space. Following introduction of the agent at a first neuron, the agent is transported to second, third, and/or higher order neurons, which are in turn synaptically connected to subsequent neurons. The mechanism of transsynaptic transport includes, but is not limited to, exocytosis from the primary neuron followed by endocytosis by the secondary neuron. The exocytotic and endocytotic events may include vesicle and/or granule mediated release and uptake, with the agent incorporated within a membrane bound organelle.

In one embodiment, interneuronal transport is used to target agents into the central nervous system, to include the brain and the spinal cord, through introduction into the peripheral nervous system, to include the motor and sensory systems. In one embodiment, agent may diffuse through the perineurium that is the sheath of connective tissue enclosing a bundle of nerve fibers. For example, the optic nerve perineurium may be a conduit between the eye and the central nervous system.

In one embodiment, an agent either alone or in combination is neurally transported in a vector. Vectors, such as viruses and plasmids, are used to contain genes that are being newly introduced into a cell or cell nucleus. The genes may themselves contain a modification that will achieve an effect. The genes may be unmodified but, once introduced into a cell or cell nucleus, they may achieve an effect.

The indiscriminate location of the vector, or release of the gene contained in the vector, can induce effects at locations where such effects are not needed. One example is gene therapy to provide angiogenic agents to facilitate vessel production in patients in need of such therapy. For example, ocular pathology may cause the retinal artery to compress the retinal vein (central retinal vein occlusion, CRVO). In an attempt to compensate, the retinal vein drains through the optic nerve. However, blockage of the optic nerve reduces or prevents this blood flow, resulting in visual disturbances. In such patients, it is desirable to provide angiogenic gene therapy to treat or improve retinal vein blood flow. While useful in moderation, however, angiogenic gene therapy can result in abnormal blood vessels that bleed inside the eye. Thus, it is desirable to contain such agents at the desired site. Administration of gene vectors in the systemic circulation, or inside a cavity such as the eye, however, results in indiscriminate release of vectors in the body or inside a body cavity.

To reduce such effects, localized gene therapy is used whereby a vector transfected with the desired gene to achieve therapy is provided at a site in the peripheral nervous system for neural transport to the central nervous system where therapy is desired. As examples, a gene encoding an angiogenic factor is provided at a site where new blood vessels are desired (e.g., in the eye or brain), or a gene encoding an anti-angiogenic factor is provided at a site where it is desirable to inhibit blood vessels. The vector may be provided in or with a biocompatible substance that substantially prevents the transfected vector from leaving the specific site. The substance may be a matrix, gel, polymer, liposome, capsule, nanoparticle, and/or microparticle. In one embodiment, the substance is provided to the site in a pro-entraining form, and then forms the substance at the site where it is provided, for example, providing thrombin and fibrinogen, which then forms a fibrin entraining network in situ.

This embodiment enhances controlled localization, positioning, or placement of, for example, a vector containing a gene at an anatomical and/or physiological site where it is desirable to locate the gene(s) provided by the vector. In one embodiment, the method localizes genes that enhance neovascularization (i.e., genes encoding angiogenic agents) by establishing new anastomoses within a system, or between two or more systems. In another embodiment, the method localizes genes that inhibit neovascularization (i.e., genes encoding antiangiogenic agents) at sites where new blood vessel growth is undesirable. The method enhances control and maintenance of vector localization, so that the vector does not significantly locate its associated gene(s) or gene product(s) (e.g., angiogenic agents or anti-angiogenic agents) substantially beyond the desired specific site.

In one embodiment, a vector containing a gene encoding an angiogenic factor is neuronally provided to a site within the central nervous system or peripheral nervous system where it is desirable to provide an angiogenic factor to establish new blood flow. In one example, blood flow is established between ocular nerves and the retinal circulation and/or the choroidal circulation in a patient with central branch or nerve occlusion. In this condition, blood flow is hampered by occlusion in the pathway of the returning nerve. Selectively using a neuronal pathway to locate gene(s) encoding angiogenic factor(s) can establish or create one or more new channels between the retinal nerve and choroidal circulation that previously did not exist.

In one embodiment, the agent is provided by an ocular route other than topical ocular administration to reach the optic nerve. Examples of such ocular routes include, but are not limited to, intraocular injection and intraocular implantation. The agent may be administered by intraocular injection or intraocular implantion either alone, formulated in a matrix such as a microsphere, nanospheres, liposome, microcapsule, nanocapsule, etc., formulated for vector delivery, etc. Examples of intraocular matrices include, but are not limited to, those amenable for scleral suturing, scleral tunneling, etc. Examples of intraocular injection include, but are not limited to, intravitreal injection, subconjunctival injection, retrobulbar injection, subretinal injection, intraretinal injection, etc.

For ocular administration, potential or actual toxicity of the agent is a consideration. Toxicity concerns arise because of the higher degree of invasiveness when agents are administered by intraocular injection or intraocular implantation, compared to the same agents administered by a topical ocular route. Toxicity concerns also arise because of the low therapeutic index (the degree between a dose that is efficacious and a dose that is toxic) as a property of the agents themselves. For example, macrolides and/or mycophenolic acid act by suppressing the immune system, such that there is a narrow window between efficacy and toxicity.

In one embodiment, a biocompatible composition is intraocularly administered by a non-topical ocular route in an amount or at a dose that does not result in substantial toxicity to the eye. As used herein, a lack of substantial toxicity encompasses both the absence of any manifestations of toxicity, as well as manifestations of toxicity that one skilled in the art would consider not sufficiently detrimental to decrease or cease treatment. As one example, fibrin deposits may be present indicating some toxicity, but less than substantial toxicity if their duration, number, etc., does not warrant that treatment be curtailed or stopped. As another example, white vitreous bodies and fibrin bodies may be present indicating some toxicity, but less than substantial toxicity if their duration, number, etc., does not warrant that treatment be curtailed or stopped.

As one example, a dose up to about 200 μg Cyclosporin A may be intraocularly injected without substantial toxicity to the patient. As other examples, the following macrolides at the stated doses may be intraocularly injected without substantial toxicity to the patient: up to about 20 μg sirolimus (rapamycin), up to about 200 μg clarithromycin, and/or up to about 1 mg clindamycin. As another example, an implant may contain a macrolide formulated to release a daily dose that does not exceed about 40 μg/ml per day in one embodiment, or to release a daily dose from about 10 μg/ml per day to about 30 μg/ml per day in another embodiment. As an example of intraocular injection of a steroid without substantial toxicity, betamethasone (Celestone®) at a dose up to about 20 mg, and/or tobramycin/dexamethasone (TobraDex®) at a dose up to about 4 mg may be used. As an example of an antibiotic that may be intraocularly injected without substantial toxicity to the patient, doxycycline at a dose up to about 150 μg, tobramycin at a dose up to about 200 μg, amphtotericin B at a dose up to about 10 μg, ampicillin at a dose up to about 500 μg, clarithromycin at a dose up to about 200 μg, clindamycin at a dose up to about 1 mg, erythromycin at a dose up to about 0.5 mg, fluconazole at a dose up to about 0.002 mg, gatifloxacin at a dose up to about 0.15 mg, ketoconazole at a dose up to about 0.002 mg, and/or vancomycin at a dose up to about 2 mg may be used. As an example of an anti-viral agent that may be intraocularly injected without substantial toxicity to the patient, ganciclovir at a dose up to about 0.4 mg may be used, and/or foscarnet at a dose up to about 2 mg may be used. As an example of an anti-proliferative agent that may be intraocularly injected without substantial toxicity to the patient, 5-fluorouracil at a dose up to about 1 mg may be used. As an example of an anti-angiogenesis agent that may be intraocularly injected without substantial toxicity to the patient, bevacizumab (Avastin®) at a dose up to about 5 mg, ranibizumab (Lucentis®) at a dose up to about 3 mg, and/or pegaptanib (Macugen®) at a dose up to about 0.3 mg may be used.

In another embodiment, the agent is provided inside the retina in approximation to a major branch of the central retinal vein at the junction of the retina and choroid. Selective localization of a vector containing a gene encoding an angiogenic factor(s) at such location enhances creation of choroid-retinal anastomoses. Thus, a controlled collateral route of blood flow is established to provide oxygen, nutrients, etc. from the blood where such a flow did not previously exist.

In one embodiment, the method is used to establish non-ocular neovascularization. As one example, the method can be used to localize vectors containing gene(s) encoding vasculogenic and/or angiogenic agent(s) at a site or sites along the peripheral nervous system and/or central nervous system where new blood vessel formation needs to be stimulated (e.g., patients with peripheral nerve disease) such that new vessels are desirable. As another example, the method can be used to localize these vectors in brain to provide new vessels in patients with cerebral ischemia. Such treatment is beneficial for patients experiencing or at risk for ischemia due to perturbations in vascular supply to the brain. Ischemia can occur as a result of a single or multiple small infarctions (e.g., stroke), a transient ischemic attack (TIA), traumatic brain injury (TBI), or atherosclerosis. The patient may experience dementia if ischemia occurs in a region of the brain controlling thought and memory processes, and agents that reduce or prevent sequelae of ischemia are desirable. For example, one or more agents that affect nitric oxide (NO) synthase, such as arginine hydrochloride, may be administered. Arginine hydrochloride reduces or prevents the decline in organ function following an ischemic episode.

In one embodiment, the method is used to localize vectors containing gene(s) encoding a neurotrophin such as nerve growth factor-β (NGFβ), brain-derived neurotrophic factor (BDNF), neurotrophin 3 (NT-3), neurotrophin 4 (NT-4), neurotrophin 6, ciliary neurotrophic factor (CNTF), or glial cell derived neurotrophic factor (GDNF), and/or a neuropoietic factor such as leukemia inhibitory factor (LIF), ciliary neurotrophic factor (CNTF), oncostatin M, growth-promoting activity, or cardiotrophin 1. These factors may, for example, be disseminated to retinal pigment epithelial cells to inhibit the progress of retinitis pigmentosa.

In one embodiment, the inventive method enhances agent containment by providing a material or substance by which or in which the vector is contained or retained along a neural conduit. The material or substance is any biocompatible material that will retain, entrain, encapsulate, and/or contain the vector as it is transported. In one embodiment, the method provides controlled release of the contained agent.

Vectors that may be used include viral vectors. Non-limiting examples of viral vectors are known to one skilled in the art and include adenovirus, recombinant adenovirus, adeno-associated virus (AAV), lentiviruses, retrovirus, alphavirus, etc. Vectors that may be used also include non-viral gene delivery vectors. Non-limiting example of non-viral gene delivery vectors are known to one skilled in the art and include naked DNA, polycation condensed DNA linked or unlinked to killed adenovirus, small interfering RNAs (siRNAs, e.g., SeqWright Inc., Houston Tex.), etc.

In one embodiment, vectors that contain the gene of interest are provided with a substance that will not significantly spread or migrate after injection. They may be mixed into the substance, or may be provided essentially simultaneously with the substance. In one embodiment, the substance is one or more of a natural and/or synthetic semisolid, gel, hydrogel colloid, reticular network, matrix, etc. In one embodiment, the substance forms in situ. In one embodiment, the substance is a hydrogel liquid below body temperature, but gels to form a shape-retaining semisolid hydrogel at or near body temperature. In one embodiment, the substance is polyethylene glycol (PEG). In one embodiment, the substance is one or more of polyanhydrides; polyorthoesters; polylactic acid and polyglycolic acid and copolymers thereof; collagen; protein polymers; polymers, copolymers, and derivatives of polyester, polyolefin, polyurethane, polystyrene, polyethylene glycol/polyethylene oxide, polyvinylalcohol, etc.

In one embodiment, the substance is a combination of fibrinogen and thrombin that, when mixed, forms a reticular or network structure (e.g., a fibrin network). As known to one skilled in the art, the structure of fibrin may be altered by varying the concentration of thrombin mixed with fibrinogen. Relatively lower thrombin concentrations produce relatively thicker fibrin fibrils with a larger pore size, slower setting rate, and slower degradation rate. Thus, the substance may be altered to contain a vector for a desired duration and with a desired durability, delivery rate, degradation rate, geometry, etc., as known to one skilled in the art. The vector(s) may be mixed with either fibrinogen and/or thrombin and injected together to create a vector entrapped inside the mesh of fibrin. Containment of the vectors at a desired site enhances control of the gene product, for example, by reduced spreading immediately after administration (e.g., injection, implantation, etc.).

The method may be used for delivering a vector containing any gene to inhibit or promote a process only at a defined physiological or anatomical location, e.g., at or in a defined area or tissue. The method may also be used for modifying release over time to provide sustained or controlled release. An extended release formulation is also termed a controlled release formulation, formulated so that the release of the agent occurs in an extended or controlled fashion in contrast to, for example, a bolus introduction. An alternative embodiment is a delayed release formulation, formulated to minimize or prevent the agent located at a site other than a desired site. Both extended release forms and delayed release forms are termed modified release forms.

In one embodiment, vectors are entrained in a microencapsulated form. Examples include liposomes, microspheres, microcapsules, etc. In one embodiment, vectors are contained in particles produced through nanotechnology. Examples include soft absorbent nanoparticles, and nanoparticles with rigid shells. Other examples may be a polyvinyl alcohol hydrogel with a diameter in the range of about 500 nm to about 750 nm; a poly-N-isopropylacrylamide hydrogel (50 nm to 1 μm); a copolymer of poly(ethylene oxide)-poly(L-lactic acid); or poly(L-lactic acid) coated with poly(ethylene oxide). In another embodiment, the entrainment substance is a reservoir or depot for the vectors within an anatomical or physiological site.

When the agent is a peptide, it may be conjugated with one or more moieties that assist in axon transport and thus facilitate the endogenous transport mechanism. This is also the mechanism by which certain viral particles may be transported to the central nervous system, being used as vectors to transport appropriate therapeutic genes.

Moieties that are capable of neuronal transport include, but are not limited to, those that interact with the endogenous transport machinery including dynein, kinesin, and myosin. As one example, small consensus binding sequences of 10-25 amino acids from the binding partners of the dynein light chains Tctex-1 and 8 (LC8) facilitate interaction between the agent and dynein. A peptide that is based on either Tctex-1 or LC8 binding peptide sequences can link a peptide agent to dynein and thus facilitate neuronal transport. As another example, the nuclear localization sequence (NLS) from the protein importin, also known as karyopherin, is another moiety that can that can link a peptide agent to dynein and thus facilitate neuronal transport. Transport-facilitating moieties can also include those that interact with endogenous agents for endogenous transport.

Interneuronal transport of a peptide agent can also be facilitated by conjugating, using methods known to one skilled in the art, the peptide agent to a moiety that is capable of transsynaptic transport. These moieties include, but are not limited to, cholera toxin B subunit (CTB), tetanus toxin C fragment (TTC), lectins (carbohydrate binding moieties such as wheat germ agglutinin (WGA), neurotrophins such as nerve growth factor (NGF), brain-derived neurotrophic factor (BDNF), and the neurotrophins NT-3, NT-4/5 and NT-6), and neurotrophic viruses that include α-herpes viruses such as herpes simplex type 1, pseudorabies viruses, and rhabdoviruses. For example, the peptide agent can be operationally coupled to the tetanus toxin C fragment. Alternatively, the genetic material encoding the peptide agent can be incorporated within a virus capable of transsynaptic transport, such as a pseudorabies virus.

If the agent is a small molecule, the agent may be conjugated to an organic mimetic that facilitates agent transport. As an example, a mimetic modeled after the NLS of the HIV-1 matrix protein may be used, as known to one skilled in the art.

In one embodiment, the agent is entirely or partially contained in a microsphere, and the microsphere is transported using the microtubule system. In one embodiment, the microsphere is biodegradable and releases the agent as it degrades. In another embodiment, the microsphere is fabricated with controlled release properties (e.g., slow release, sustained release, delayed release, etc.). The microsphere may have moieties conjugated to its outer surface to facilitate transport intraneuronally and interneuronally, as previously described.

In one embodiment, a dye that diffuses along the length of the axon may allow visualization of an axon or dendrite. A high concentration of dye is directly injected into the neuronal process through a micropipette.

Agent may be introduced via invasive, minimally invasive, or non-invasive routes. Without being bound by a specific mechanism, a topical route of administration, even when coupled with a facilitating mechanism or compound, may be less desirable than a more invasive route of administration due to, for example, neuron proximity or other factors. In one embodiment, the agent is introduced into the eye by an ocular route. Examples include, but are not limited to, intraocular injection (e.g., subconjunctival, retrobulbar, subretinal, intraretinal, intravitreal), topical administration (e.g., liquid drops, ointment, cream), ocular implantation, trans-scleral delivery, etc. The agent may be injected directly into and/or adjacent a nerve root, nerve fiber or bundle such that it is neuronally disseminated, e.g., into or adjacent the sphenopalatine ganglion. The agent may be injected into dorsal root ganglion for transport of agent to the somatosensory system. The agent may be injected into regions of the optic nerve or retina for transport to the visual system, to other components of the peripheral nerve system, or to the central nervous system.

In one embodiment, the agent is introduced at one or more sites in the peripheral nervous system that are used as acupuncture sites. Administration methods may include, but are not limited to, subcutaneous injection, topical administration, transdermal administration, any of which may be either non-facilitated or facilitated. Facilitated administration includes the use of electrical current (e.g., iontophoresis), thermal energy (e.g., heat), ultrasound energy, radiant energy (e.g., laser, infrared, near-infrared, mid-infrared), etc. to disseminate agent to the desired site, at the desired interval, etc.

Most acupuncturists use traditionally identified points mapped to 14 major meridian lines, one meridian for each of the 12 inner organs, one meridian along the spine (called the governing vessel), and another along the midline of the abdomen (called the conception vessel). However, the number of points identified by acupuncturists has vastly increased. There are extra meridians (some of them outlined in ancient times, others modern) with their own sets of points, there are special points (off meridians), and there are complete mappings of body structures and functions by points along the outer ears, on the nose, in the scalp, on the hands, on the feet, and at the wrists and ankles.

As non-limiting examples, the following are known acupuncture points that can be used according to the inventive method. A large intestine meridian point is located on the back side of the hand between the thumb and first finger. Another key point on this meridian is located at the elbow. A lung meridian point is located above the wrist on the inside of the arm. A stomach meridian point is located on the front of the leg, just below the knee. Many clinical trials have been conducted with treatment of this point, demonstrating positive effects in treating anemia, immune deficiency, fatigue, and numerous diseases. A spleen meridian point is located on the inner side of the leg just above the ankle. Another key point on this meridian is located just below the knee. A gallbladder meridian point is located at the base of the skull where it joins the neck in back. Another key point on this meridian is located on the outer side of the knee. A liver meridian point is located on the top of the foot, between the first and second toes. The adjacent point in the meridian, at the webbing between the toes, is also considered important and is frequently needled along with the aforementioned site. A pericardium meridian point is located on the inner arm, just above the wrist. A heart meridian point is located on the outer side of the wrist. A urinary bladder meridian point is located at the back of the knee. Another important point on the bladder meridian is in the lumbar area (hip level) near the spine. A large section of the bladder meridian is stated to be of importance because, as it flows along either side of the spine (in two parallel lines on each side), it associates with the internal organs in the vicinity. A kidney meridian point is located just behind the inner ankle. A point located on the outer side of the arm, above the wrist, is considered to be a point for special type of organ system that spans the entire torso and is mainly used in treatment of disorders along the pathway of this meridian, that is, of the fingers, hand, arms, neck, ears, cheek, and top of the head. A small intestine meridian point is located on the side of the hand, below the little finger. A governing vessel point is located at the top of the head. Another key point on this meridian is located just below the seventh cervical vertebrae (shoulder level).

In one embodiment, a device may release the agent by electromotive administration, also referred to as iontophoresis, using a small electrical current passed through the nerve from the point of agent administration or delivery. For example, for therapy of an ocular pathology, an ocular agent may be injected into the vitreous cavity, and then diffusion to the optic nerve may be assisted using iontophoresis. In this embodiment, the device contains an electrode, i.e., an anode and/or cathode depending upon the charge state of the agent(s). The device may contain both anode and cathode to accommodate different agents contained in different compartments of the device. An electrode of opposite polarity (cathode and/or anode) is inserted at a site opposite the device. For example, one electrode may be located on a contact lens inserted in the eye, and the other electrode may be positioned at the area of the occipital lobe, the visual processing center of the brain located at the back of the skull.

The flow of current from the point of administration through the nerve is regulated externally by an energy source. When current is applied, an electrical potential difference is generated between the two electrodes, facilitating agent transport through the nerve. For example, the eyelid is maintained opened and the contact lens is applied, then current is applied to stimulate agent translocation through the cornea, anterior chamber, sclera, and vitreous. Such administration may permit a relatively higher concentration of agent to be delivered diffusively at a site requiring agent. The dose of agent delivered depends upon the current and duration selected. In one embodiment, a current between about 0.5 mA and about 4 mA is applied for between a few seconds to about 20 min. Iontophoresis delivery itself has no side effects and there is no pain associated with agent administration. Thus, it may be used in any embodiment.

For use with ultrasound, a water soluble gel is applied to the skin on and surrounding the area to be treated with ultrasound radiation. The source of ultrasound energy is set at the desired level of intensity, for example, 12.5 W, with the timer set for fifteen minutes. A transducer is gently placed on the prepared area and sonic energy is applied using continual movement of the transducer in either a clockwise or counterclockwise direction, limiting the area to a circle of about 1-12 inches in diameter. Consistent pressure is applied over the area.

Bioelectromagnetic energy may also be used, for example, using applied pulsed and direct current electromagnetic fields. Application of electromagnetic fields may be used for nerve stimulation (transcutaneous, transcranial, neuromagnetic, electromyography, electroencephalography, electroretinography, and low energy emission therapy), wound healing in soft tissues (skin and vasculature), electroacupuncture to relieve pain such as post operative pain, tissue regeneration (particularly nerve regeneration), and stimulation of the immune system through changes in calcium transport and mediation of the mitogenic response. Bioelectromagnetic applications have also been used in treating osteoarthritis.

In one embodiment, the agent is introduced at a non-ocular sensory site. As one example, the agent may be introduced in an area with a high concentration of nerve endings such as the tongue or ear. As another example, the agent may be nasally introduced by inhalation and may access a number of nerve terminals in the nose. Agent absorption at the olfactory region of the nose provides a potential for agent availability to the central nervous system.

Agent absorption is influenced by the residence (contact) time between the agent and the epithelial tissue. Mucociliary clearance is inversely related to the residence time and therefore inversely proportional to the absorption of agents administered. Residence time in the nasal cavity may be prolonged by using bioadhesive polymers, microspheres, chitosan or by increasing the viscosity of the formulation. Formulations for intranasal administration include agents in solutions, suspensions, and/or emulsions administered as drops, sprays, or aerosols, and gels and/or ointments administered by application to the mucosa or squirting into the nose.

As another example, the agent may be introduced into the Eustachian tube to access the inner ear and/or brain. The Eustachian tube is a membrane-lined tube that connects the middle ear to the back of the nose (“throat” or pharynx). The pharynx extends from the base of the skull to the level of the sixth cervical vertebra. Inferiorly, it opens into the larynx (respiratory system) and esophagus (digestive system). The pharynx is divided into the nasopharynx, oropharynx, and laryngopharynx. The nasopharynx is the portion of the pharynx that is posterior to the nasal cavity and extends inferiorly to the uvula. The oropharynx is the portion of the pharynx that is posterior to the oral cavity. The laryngopharynx is the most inferior portion of the pharynx that extends from the hyoid bone down to the lower margin of the larynx.

Because of anatomy, agent can access the Eustachian tube by administration into either the nose or the pharynx. In one embodiment, agent may be administered into the nose, e.g., formulated as an inhalable, a spray, a topical, etc. as a route from the nose to the Eustachian tube. In another embodiment, agent may be administered into the mouth, e.g., formulated as an inhalable, spray, aerosol etc. for spraying or breathing into the mouth (not entering the lungs), as a route from the pharynx to the Eustachian tube. Agent can then exit the body by simple exhalation through the nose and/or mouth. In these embodiments, agent has access via the Eustachian tube to the middle ear, inner ear, and brain via the eighth cranial nerve (vestibulocochlear nerve).

In one embodiment, agents formulated with or in microspheres provide more prolonged contact with the nasal mucosa and thus enhance absorption. Microspheres for nasal applications have been prepared using biocompatible materials, such as starch, albumin, dextran and gelatin (Bjork E and Edman P., Microspheres as nasal delivery system for peptide drugs. J. Controlled Release 21, 165 (1992), which is expressly incorporated by reference herein).

In embodiments using a type of facilitated transport such as a vector, iontophoretic delivery, etc., the concentration of agent may be lower than in embodiments where such transport is not facilitated, because of directed or facilitated transport that results in a higher concentration of agent reaching the desired site. In another embodiment, a supratherapeutic but non-toxic dose of agent may be administered in an area adjacent the site of administration.

Administration may be intermittent, sustained for a particular duration, as needed, to achieve a desired effect, etc. Multiple administrations of agent may be used. An agent may be formulated to be taken up by the neuron by receptor-mediated endocytosis if the agent is conjugated to a suitable moiety, such as a ligand for a particular receptor. Receptor mediated endocytosis is a process by which cells internalize molecules or viruses. It requires ligand interaction with a specific binding protein, a receptor, on or in the cell membrane. Ligands that are internalized by receptor-mediated endocytosis include, but are not limited to, toxins and lectins such as diphtheria toxin, pseudomonas toxin, cholera toxin, ricin, and concanavalin A; viruses such as Rous sarcoma virus, Semliki forest virus, vesicular stomatitis virus, and adenovirus; serum transport proteins and antibodies such as transferrin, low density lipoprotein, transcobalamin, IgE, polymeric IgA, maternal IgG, and IgG, via Fc receptors; and hormones and growth factors such as insulin, epidermal growth factor, growth hormone, thyroid stimulating hormone, nerve growth factor, calcitonin, glucagon, prolactin, luteinizing hormone, thyroid hormone, platelet derived growth factor, interferon, and catecholamines. An example of agent internalization into a cell by receptor-mediated endocytosis is the conjugation of transferrin with therapeutic drugs, proteins, or genetically by infusion of therapeutic peptides or proteins into the structure of transferrin. Also, conjugation of the agent to the OX26 monoclonal antibody which recognizes the transferrin receptor may be used to deliver therapeutic agents inside the cell via receptor-mediated endocytosis.

Alternatively, the agent may be introduced into the cell by incorporating the agent within liposomes. As known to one skilled in the art, liposomes are vesicles surrounded by a lipid membrane resembling that of a cell and are endocytosed by the cell. Examples of liposome-mediated uptake include nucleic acids, proteins, and agents.

Pathologies for which the inventive method may be used include, but are not limited to, the following. Ocular pathologies such as age related macular degeneration, retinitis pigmentosa, diabetic retinopathy, scleritis, uveitis, vasculitis; ocular cancers such as retinoblastoma, choroidal melanoma, pre-malignant and malignant conjunctival melanoma; and pathologies related to the optic nerve. Neurodegenerative diseases including but not limited to Parkinson's disease and Alzheimer's disease. Inflammatory processes including but not limited to multiple sclerosis and arthritic anterior ischemic optic neuropathy. Neurological pathologies including but not limited to epilepsy, narcolepsy, seizures, CNS pathologies, and spinal cord injuries.

The optic nerve, which connects the eye with the brain, is a continuation of the axons of the ganglion cells in the retina. There are about 1.1 million nerve cells in each optic nerve. Optic atrophy of the optic disk results from degeneration of the nerve fibers of the optic nerve and optic tract. It can be congenital (usually hereditary) or acquired. If acquired, it can be due to vascular disturbances (occlusions of the central retinal vein or artery or arteriosclerotic changes within the optic nerve itself), may be secondary to degenerative retinal disease (e.g., optic neuritis or papilledema), may be a result of pressure against the optic nerve, or may be related to metabolic diseases (e.g., diabetes), trauma, glaucoma, or toxicity (e.g., alcohol, tobacco, etc.). Degeneration and atrophy of optic nerve fibers is irreversible and results in loss of vision.

Optic neuritis is an inflammation of the optic nerve. It may affect the part of the nerve and disk within the eyeball (papillitis), or it may affect the part behind the eyeball (retrobulbar optic neuritis). It also includes degeneration or demyelinization of the optic nerve. It can be caused by demyelinating diseases (e.g., multiple sclerosis, postinfectious encephalomyelitis), systemic infections (viral or bacterial), nutritional and metabolic diseases (e.g., diabetes, pernicious anemia, hyperthyroidism), Leber's hereditary optic neuropathy (a rare form of inherited optic neuropathy), secondary complications of inflammatory diseases (e.g., sinusitis, meningitis, tuberculosis, syphilis, chorioretinitis, orbital inflammation), toxic reactions (to tobacco, methanol, quinine, arsenic, salicylates, lead), and trauma. Papilledema is edema or swelling of the optic disc (papilla), most commonly due to an increase in intracranial pressure (often from a tumor), malignant hypertension, or thrombosis of the central retinal vein. Secondary optic atrophy and permanent vision loss can occur if the primary cause of the papilledema is left untreated.

Ischemic optic neuropathy is a severe blinding disease resulting from loss of the arterial blood supply to the optic nerve, as a result of occlusive disorders of the nutrient arteries. Glaucoma damages the optic nerve because the intraocular pressure (IOP) is higher than the retinal ganglion cells can tolerate. The cause is fluid excess, either because of increased production or decreased clearance. Glaucoma eventually results in the death of the ganglion cells and their axons that comprise the optic nerve, causing fewer visual impulses from the eye to reach the brain. In advanced glaucoma, the peripheral retina is decreased or lost, leaving only the central retina (macular area) intact, resulting in tunnel vision. If left untreated, glaucoma eventually leads to optic atrophy and blindness.

From the above description, other variations or embodiments of the invention will also be apparent to one of ordinary skill in the art. As one example, the invention may be used to facilitate growth of transplanted neuronal cells, either mature or immature, and/or stem cells in the eye or brain. As another example, other ocular routes of administration and injection sites and forms are also contemplated. As another example, the invention may be used in patients who have experienced ocular trauma, ischemia, inflammation, etc. Thus, the forgoing embodiments are not to be construed as limiting the scope of this invention.

What is claimed is:

1. A method of disseminating a biocompatible agent to a patient, the method comprising providing to a patient in need thereof an agent capable of exerting an effect at a distal neural site, the agent provided by a non-topical ocular route of administration for dissemination via a neural conduit to exert the effect at the distal neural site.

2. A method of disseminating a biocompatible agent to a patient, the method comprising providing to a patient in need thereof at a first peripheral nervous system site, an agent capable of exerting via dissemination by a neural conduit an effect at least one of a second peripheral nervous system site or a central nervous system site, the agent administered at least one acupuncture site by at least one of injection, transdermal administration, or facilitated topical administration.

3. A method of disseminating a biocompatible agent to a patient, the method comprising providing to at least one of an oral cavity or a nasal cavity of a patient in need thereof for dissemination via a neural conduit from a Eustachian tube to at least one of a second peripheral nervous system site or a central nervous system site, an agent capable of exerting an effect at the site.

4. The method of claim 1 wherein the distal neural site is a central nervous system site or a non-ocular peripheral nervous system site.

5. The method of claim 1 wherein the route of administration is at least one of intraocular injection or intraocular implantation.

6. The method of claim 1 wherein the agent is disseminated in the perineurium of the optic nerve.

7. The method of any of claim 1, claim 2, or claim 3 wherein dissemination is facilitated by application of at least one of electrical current, ultrasound energy, radiant energy, bioelectromagnetic therapy, or thermal energy.

8. The method of any of claim 1, claim 2, or claim 3 wherein the agent is at least one of a drug, a vaccine, a peptide, a protein, or a vector containing a gene therapy agent.

9. The method of any of claim 1, claim 2, or claim 3 wherein the agent is conjugated to a transport facilitating moiety.

10. The method of any of claim 1, claim 2, or claim 3 wherein the agent is formulated for controlled release.

11. The method of any of claim 1, claim 2, or claim 3 wherein the agent is selected from at least one of a macrolide, anti-prostaglandin, matrix metalloproteinase inhibitor, anti-viral agent, antioxidant, anti-cell migration agent, angiogenic agent, anti-angiogenic agent, or anti-neoplastic agent.

12. The method of any of claim 1, claim 2, or claim 3 wherein the agent is for alleviation of at least one of retinitis pigmentosa, age related macular degeneration, arthritic anterior ischemic optic neuropathy, multiple sclerosis, diabetic retinopathy, scleritis, uveitis, vasculitis, retinoblastoma, choroidal melanoma, pre-malignant and malignant conjunctival melanoma, optic nerve pathologies, Parkinson's disease, Alzheimer's disease, epilepsy, narcolepsy, seizures, spinal cord injury, or central nervous system pathologies.

13. The method of any of claim 1, claim 2, or claim 3 wherein the agent is formulated as at least one of a solution, suspension, emulsion, microspheres, nanospheres, liposomes, microparticles, or nanoparticles.

14. A method of disseminating a biocompatible agent, the method comprising providing to an individual at a first neural site an agent selected from the group consisting of an acetylcholinestrase inhibitor, an L-type calcium channel modulator, an agonist of a nicotinic alpha-7 receptor, an inhibitor of phosphodiesterase 10, an inhibitor of phosphodiesterase 4, and combinations thereof, the agent disseminated along a neural conduit to a central nervous system site in need of therapy, the agent administered by at least one of
a non-topical ocular route,
injection, transdermal application, or facilitated topical administration at least one acupuncture site,
administration at an olfactory site, or
pharynx or nasal administration to a Eustachian tube.

15. The method of claim 14 wherein the agent is targeted to a region of the brain.

16. The method of claim 14 wherein administration is facilitated by application of at least one of electrical current, ultrasound energy, radiant energy, bioelectromagnetic therapy, or thermal energy.
